(19) United States
(12) Patent Application Publication
(10) Pub. No.: US 2004/0151724 A1
(43) Pub. Date: Aug. 5, 2004

(54) Antibody fab fragments specific for breast cancer

(76) Inventor: Julia Coronella-Wood, Carlsbad, CA (US)

Correspondence Address: QUARLES & BRADY STREICH LANG, LLP, ONE SOUTH CHURCH AVENUE, SUITE 1700, TUCSON, AZ, 85701-1621, US

(21) Appl. No.: 10/698,041

(22) Filed: Oct. 30, 2003

Related U.S. Application Data
(60) Provisional application No. 60/423,052, filed on Oct. 31, 2002.

(51) Int. Cl. A61K 39/395; C12Q 1/68; C07H 21/04; C07K 16/30; C12N 5/06

(52) U.S. Cl. 424/155.1; 530/388.8; 536/23.53; 435/6; 435/69.1; 435/320.1; 435/344

(57) ABSTRACT

Human antibody fragments (Fab 14.6.19 and Fab 14.6.20) including polynucleotides and amino acids (SEQ ID NO: 3, SEQ ID NO: 4, SEQ ID NO: 5 and SEQ ID NO: 6) that identify them. Both Fabs are fully human, are affinity matured in vivo, are highly specific for breast cancer, and target an antigen that is immumogenic in vivo. Thus, each Fab may be a useful clinical reagent for diagnosis or therapy of breast cancer and may also lead to the discovery of a novel immunogenic and tumor specific breast cancer antigen.

### CROSS-REFERENCE TO RELATED APPLICATIONS

This application claims priority to U.S. Provisional Application No. 60/423,052, filed on Oct. 31, 2002.

### BACKGROUND OF THE INVENTION

1. Field of Invention

This invention generally relates to the field of cancer biology and in particular to novel breast-cancer specific antibody fragments, polypeptides, and polynucleotides encoding those fragments.

2. Description of the Related Art

Breast cancer is the most common life-threatening malignancy diagnosed in women. Approximately 192,000 new cases of breast cancer were diagnosed in the United States in 2001 and roughly half of these cases occurred in women over the age of 65. Age is the single greatest risk factor for developing breast cancer. Despite the common misconception that breast cancer in the elderly is a benign disease, breast cancer kills approximately 24,000 women over 65 each year. The elderly account for nearly 60% of all deaths due to breast cancer. Because of their comorbidities, socioeconomic status, and limited resources, many older patients receive inferior therapy and consequently have decreased survival rates.

In addition, meta-analysis of patients with early breast cancer suggests standard chemotherapies are not as effective in older women as in younger women. In fact, a recent study shows there was no statistically significant benefit seen to receiving adjuvant therapy for women over age 70. These results may be skewed by the under-representation of elderly women who participate in or are offered participation in clinical trials. Still, the results suggest that breast cancer in elderly patients may respond differently to anti-tumor therapies.

Novel therapies that produce less toxicity, maintain quality of life and physical function, and target their breast cancer specifically are needed. Unfortunately, aromatase inhibitors are associated with elevated risk for decreased bone mass, while tamoxifen is associated with increased risk of thromboembolism, both issues of particular concern in the geriatric population.

Biologic therapies can be uniquely designed to target individual tumors with minimal toxicity. Rituxan®, an antibody therapy directed against a B cell antigen, has been found to increase response rates and survival in elderly patients with aggressive non-Hodgkin's lymphoma. Herceptin, an antibody targeting the her2/neu receptor on breast cancer cells has also been found to be well-tolerated and effective in elderly breast cancer patients. Unfortunately, overexpression of the her2/neu receptor is seen in less than 20% of elderly breast cancer patients. Consequently, this well tolerated and effective therapy can be offered to only a small subset of elderly patients. Different antigenic targets are required for elderly patients as their tumors often express different proteins and receptors than tumors arising in younger patients. Determining the antigens expressed in breast cancer of the elderly may improve understanding of the biology and natural history of the disease in this subset of patients and could lead to the development of new diagnostic and therapeutic reagents.

Decreasing systemic cytotoxic immunity with age has been well documented, impairing the potential efficacy of cell based immunotherapy in the elderly. The proliferative potential of T cells in response to antigens challenge decreases with age, as does the ability to generate cytotoxic precursors. However, ADCC remains unimpaired with aging, suggesting that antibody immunotherapy will have equal efficacy in elderly patients. Although this area has received little attention, murine studies indicate that young and aged animals have equal NK and ADCC activity. As far as is known, no studies specifically addressing this issue have been published to date, but anecdotal evidence suggests that trastuzumab is effective in the minority of elderly patients with Her-2/neu positive tumors. Further, rituximab has also demonstrated efficacy in older patients, indicating functional ADCC in the elderly in vivo. Therefore, an antibody targeting a more universally expressed breast cancer antigen could be useful.

Heterogeneity predominates in reports of changes in humoral immunity with aging. However, there are a number of reports of distinct changes in humoral immune responses with age. Production of antibody in response to foreign antigen decreases with age, but there is no change in overall numbers of lymphocytes. There is also a decrease in affinity and avidity of response that may be due to reliance upon natural antibody or memory responses in older patients rather than adaptive immunity. For example, one research group found decreased rates of hypermutation in kappa light chains in patients of average age of 83 as compared to an average age of 21. In aged mice, the number of B cell precursors decreases, supporting the hypothesis of a decreased ability to mount new adaptive responses. However, overall levels of VH hypermutation in peripheral blood are equivalent in young and elderly adults. In older humans, there is a repertoire shift to use of VH4 Ig gene family members, while VH3 dominates in younger patient repertoires. There is also an apparent shift away from high-affinity IgG1 responses in older patients challenged with influenza vaccination. There is an increase in the presence of low affinity autoantibodies. Taken together, these data suggest that the antibody repertoire may be more limited in older individuals, but is not clear that this translates into impaired function.

Isolation of fully human affinity-matured antibodies to tumor-specific cell surface antigens has proven problematic. Most breast cancer antigens discovered by serum antibody responses are intracellular, and not specific to breast tumors, and are thus of limited utility. Antigens against which a serum antibody response occurs include p53, c-myc, and c-myb. Cell surface proteins are often poorly immunogenic for a number of reasons including shedding, glycosylation and low copy number. Possible exceptions to this are Her-2/neu and MUC-1, which are overexpressed on breast tumors, and which are weakly immunogenic in vivo.

Her-2/neu is overexpressed on 25-50% of breast tumors as the result of gene amplification, and overexpression is correlated with poor prognosis. Less than 20% of geriatric breast tumors overexpress Her-2/neu. In the subset of patients with Her-2/neu overexpression, 20% produce serum antibodies against Her-2/neu. The immunogenicity of Her-2/neu may result from overexpression in an ectopic tissue, in conjunction with immunological “danger” signals including tumor necrosis. Although Her2 is expressed on cardiac tissue, and so is not completely tumor-specific, anti-Her-2/neu antibody immunotherapy with trastuzumab has produced excellent response rates with minimal toxicity both alone and in combination chemotherapy.

MUC-1 is immunogenic in an underglycosylated form occurring on tumors. There is evidence that a naturally occurring humoral response to MUC-1 can be protective in breast cancer. The presence of serum MUC1/antibody complexes correlates with recurrence-free survival. However, clinical trials targeting MUC1 in breast cancer have thus far produced modest results. The Theratope® vaccine (Biomira, Inc.) consists of the mucin Sialyl-Tn conjugated to Keyhole Limpet Hemocyanin. In phase II trails of Theratope, patients with high antibodies titers against Theratope showed a trend towards prolonged survival.

Anti-actin antibodies have been cloned from breast tumor infiltrating cells in the histologic subtype medullary carcinoma. While actin is not a cell surface protein, proteolyzed actin peptides are displayed on the surface of apoptotic typical medullary carcinoma cells in vivo. However, it is not clear what the significance of this might be to other histologic types of breast cancer, as typical medullary carcinoma has a number of anomalous immunological features in comparison to non-medullary breast cancers including high levels of apoptosis, elevated HLADR expression by tumor cells, the presence of a diagnostic plasmacytic infiltrate, and elevated expression of MIB1, ICAM1 and LFA1. Recent data do not indicate production of anti-actin antibodies in infiltrating ductal carcinoma, the most common histologic type of breast cancer.

Prior to the recent discovery of breast tumor-specific TIL-B and intratumoral germinal centers, little was known about breast tumor infiltrating B cells. However, one study found that anti-tumor antibodies were produced by tumor-infiltrating B cells in approximately 70% of non-breast tumors examined. Tumors from which tumor-specific TIL-B have been cloned include melanoma, colon carcinoma, ovarian carcinoma, lung carcinoma, glioma, sarcoma, neuroblastoma, and Hodgkin's lymphoma.

Many breast adenocarcinomas contain lymphocytic infiltrates to varying degrees, with heavy infiltrates occurring in ˜20%, and moderate infiltrates in ˜50%. The composition of breast tumor-infiltrating lymphocytes (TIL) varies between patients, and is generally heterogeneous, containing both CD4 and CD8 T cells, with fewer numbers of B cells, macrophages, and NK cells. Approximately 24% of breast adenocarcinomas contain B cells, with B cells comprising up to 40% of the TIL. When present, B cells occur exclusively in follicle-like aggregates, which is consistent with in situ antigen-driven expansion. It was recently demonstrated that these aggregates are in fact functional ectopic germinal centers.

CD1a-+ dendritic cells are also a component of infiltrates, and were found in the tumor bed of all breast cancer samples examined in one study. Intratumoral dendritic cells are closely associated with tumor cells, consistent with a possible role in antigen presentation. In some cases, T cells are clustered around mature dendritic cells in peritumoral areas, which is characteristic of an ongoing immune reaction.

Geriatric breast cancer is notable for indolent growth, expression of the estrogen receptor, normal p53, lower proliferative rates, but no expression of Her-2/neu or EGFR. Age above 60 rather than menopausal status distinguishes this group from others. Expression profiling and immunohistochemical studies suggest that these Her-2/neu negative, ER positive tumors frequently observed in older women may represent a distinct subgroup with different antigen expression, clinical progression, and possibly cell of origin. Human breast ductal tissue is composed of luminal epithelium and myoepithelium, which have distinct immunohistochemical and gene expression profiles. Based on gene expression profiling, two clades of breast tumors have been identified, one of which most closely resembles luminal breast epithelium. The luminal epithelium-like tumors are largely ER positive and Her-2/neu negative.

In contrast, the more aggressive breast cancers observed in younger women represent a subset of breast cancers with distinct clinical and prognostic features including poor differentiation, high proliferative index, low estrogen receptor expression, frequent gene alterations, expression of Her-2/neu, altered p53 and host immune response. Microarray expression studies confirms this aggressive subset, and further indicates a distinct pattern of expression consistent with breast basal endothelial cells in contrast to the more indolent ER-positive breast cancers of older women which more closely resemble breast luminal epithelium.

While lymph node or spleen germinal center reactions are the paradigm for B cell expansion, B cells can proliferate and in some cases undergo affinity maturation in ectopic germinal centers, although this has previously been described only in autoimmunity. Extranodal B cell proliferation has been characterized in rheumatoid arthritis, multiple sclerosis, Sjogren's syndrome, and Grave's disease. In these cases, extranodal expansion of B cells reactive with an autoantigen has led to a pathogenic disease state. In the case of rheumatoid arthritis, “rheumatoid factor” B cells form germinal centers in the synovium, undergo somatic mutation, affinity maturation by selection, receptor revision, clonal expansion and differentiation to plasma cells. This suggests that mechanisms to delete autoreactive B cells may be deficient in ectopic germinal centers. Moreover, it was recently discovered that an analogous situation exists in breast cancer, where ectopic germinal centers reside in the tumor bed and produce tumor-specific antibodies.

The relevance to this study of an in situ tumor antigen-driven B cell proliferation in ectopic germinal centers is not its role in tumor progression but isolation of tumor-reactive and possibly high-affinity immunoglobulins. Like those observed in autoimmune disease, ectopic germinal centers in breast cancer may be capable of producing antibodies against cell surface autoantigens to a degree not allowed in lymph node follicles. The repertoire of TILB is also very limited and enriched for tumor reactive antibodies, facilitating the isolation of these antibodies without the need for negative selection. Antibodies cloned from tumor infiltrating B cells that are specific to breast cancer cell surface antigens supports the hypothesis of immune permissiveness, and demonstrates the feasibility of cloning antibodies specific to breast tumor cell surface antigens from tumor infiltrating B cells. Cell surface antigens are normally poorly immunogenic in vivo, as demonstrated by the overwhelming bias towards cytosolic antigens in breast cancer serum antibody responses. As a result, fully human antibodies against tumor specific surface antigens are rare. Thus, intratumoral germinal centers provide a unique immunological loophole for the production of antibodies against cell surface autoantigens. Further, the produced antibodies are affinity matured, as judged by somatic mutation patterns. Non-quantitative assessment of cloned Fabs in flow cytometry with breast tumor cells is also consistent with high affinity.

Affinity maturation in ectopic germinal centers associated with autoimmunity has been dissected at the molecular level in order to understand the etiology of autoimmune disease. Because ectopic germinal centers in solid tumors had not been described prior to a recent preliminary study, much of the relevant background in this area derives from studies of rheumatoid arthritis and other autoimmune diseases. In the absence of any compelling evidence to suggest a viral or other xenobiotic etiology for breast cancer, it is useful to view native anti-tumor immunity as an autoimmune response. Because breast tumors express few if any truly foreign antigens, any immune response must overcome the same tolerance hurdles as in a pathogenic autoimmune state. Germinal centers in the synovium of rheumatoid arthritis (RA) are formed via pathways similar to those for secondary lymphoid follicles. Lymphotoxin (LT)-(α1β2 production by both germinal center B and T cells and production of CXCL13 by local endothelial cells and fibroblasts contribute to germinal center formation. Like the germinal centers that have been described in breast tumors, synovial germinal centers in RA do not have clearly delineated light and dark zones. Instead, proliferating B cells occur in the follicular dendritic cell zone. Despite this, antibodies produced by germinal centers in both cases are affinity matured. It is thought that this lack of structural organization might be due to insufficient production of CXCL13 by follicular dendritic cells or its receptor on B cells, CXCR5. An unusual population of CD8+, CD40L+, IFN+, perforin-T cells are also absolutely required for germinal center formation in RA. These cells are not observed in tonsil germinal centers.

While it has been observed that CD8+ T cells in the germinal centers in breast tumors, no information regarding expression of these markers has been forthcoming. As in RA, the humoral response in breast tumors must overcome the normal deletion of autoimmunity. It is possible that the presence of antiapoptotic signals such as constitutive CD40L on local T cells may prevent apoptosis of autoreactive B cells, perhaps through induction of cFLIP. There is also an active component of FDC-mediated B cell apoptotic signaling via FasL that may be lacking in ectopic germinal centers, as one does not observe Fas (CD95) on TILB cells. In addition, unlike the germinal centers in RA, breast tumor germinal centers do not contain CD38hi plasma cells or CD27+ memory B cells. Blimp-1, IL-6 and XBP-1 contribute to plasma cell differentiation, while IL-2, IL-10 and CD40L contribute to entry into the plasma cell compartment.

One aim of the present invention is to explore the expression of these factors further in order to delineate differences in tumor immunobiology between young and elderly patients and to understand the development of intratumoral germinal center reactions.

The success of antibody therapy directed against Her-2/neu and the failure of other breast cancer antibody therapies directed against a variety of non-immunogenic antigens suggests that naturally immunogenic tumor antigens may be superior targets. Imunogenicity entails visibility and availability to the immune system, and is associated with varying degrees of tumor specificity. Further, immunogenic antigens can also be targeted by other means, and their identification gives important information about tumor immunobiology. While SEREX has been useful in identifying immunogenic antigens, it is time-consuming, can produce false positives, is biased towards soluble antigens, does not identify nonprotein antigens, and does not provide cognate antibodies, which are then often produced via murine technology. Because murine antibodies elicit HAMA responses that prevent multiple dosing and do not have Fc-mediated effector functions in humans, techniques have been developed to humanize antibodies. However, humanization is time-consuming, expensive, technically difficult, and can result in antibodies with altered affinities. Others use antigens discovered by SEREX or microarray to generate human antibodies in human immunoglobulin-transgenic mice, avoiding the need for humanization. Given the money and time demands associated with such methods, it is believed that a simpler, more direct approach offers great promise with considerable economy and efficiency, for example, the phage display methods of C. Barbas (Scripps Research Institute).

Use of phage display antibody libraries allows the rapid and inexpensive isolation of human antibodies reactive with antigen in a variety of formats, quantities, and can selectively isolate high-affinity immunoglobulin. Phage display allows the rapid isolation of high affinity Fabs reactive with cell surface antigens.

In B cell-mediated autoimmune diseases, in situ immunity is sometimes necessary and even sufficient to produce devastating degradation of the involved tissues. Research regarding breast cancer suggest that in situ immunity may be capable of producing responses that might be suppressed in a lymph node as part of the normal suppression of autoimmunity. Indeed, it was recently discovered the presence of ectopic intratumoral germinal centers in breast tumors. This had not been previously described in any tumor. These germinal centers are functional and produce phenotypically matured B cells and affinity matured, class-switched antibodies that are specific to breast cancer-specific cell surface antigens. In intratumoral germinal centers, naturally occurring immunity accomplishes what has been found to be exceedingly difficult in the laboratory: production of high affinity antibodies against tumor-specific cell surface antigens. In combination with phage display technology, this has been found to be a powerful tool to study the in situ immunobiology of breast tumors and to develop tumor-specific antibodies.

### SUMMARY OF THE INVENTION

There are few if any therapies or diagnostic tools specifically developed for, or specific studies of, tumor immunobiology in the elderly. Therefore, discovery of antigen/antibody pairs appropriate for use with geriatric breast cancer patients will improve understanding of the disease and contribute new diagnostic and therapeutic agents. In previous research, it was discovered that lymphocyte derived intratumoral germinal centers in geriatric breast tumors produce affinity-matured antibodies against breast tumor-specific cell surface antigens. Three phage displayed Fab libraries were cloned from breast tumor infiltrating B cells from geriatric breast cancer patients. Initial study of one library yielded three Fabs that bind highly tumor-specific cell-surface antigens, two of which have been sequenced at the polynucleotide and amino acid level.

Accordingly, in one aspect of the invention, polynucleotides encoding antibody 16.4.19 (SEQ ID NO: 1) is disclosed. In another aspect of the invention, polynucleotides encoding antibody 16.4.20 (SEQ ID NO: 2) is disclosed. Moreover, amino acid sequences for 16.4.19 (SEQ ID NO: 3 and SEQ ID NO: 4) and 16.4.20 (SEQ ID NO: 5 and SEQ ID NO: 6) are disclosed. Both of these antibody fragments represent significant progress over existing antibodies because (1) they are fully human and require no molecular modification beyond attachment to the IgGI Fc region for clinical use; (2) each Fab is affinity matured in vivo and may have high affinity for the breast cancer antigen it binds; (3) each Fab is highly specific for breast cancer; and (4) each Fab targets an antigen that is immumogenic in vivo. Thus, each Fab may be a useful clinical reagent for diagnosis or therapy of breast cancer and may also lead to the discovery of a novel immunogenic and tumor specific breast cancer antigen.

Various other purposes and advantages of the invention will become clear from its description in the specification that follows and from the novel features particularly pointed out in the appended claims. Therefore, to the accomplishment of the objectives described above, this invention consists of the features hereinafter illustrated in the drawings, fully described in the detailed description of the preferred embodiments and particularly pointed out in the claims. However, such drawings and description disclose only some of the various ways in which the invention may be practiced.

### BRIEF DESCRIPTION OF THE DRAWINGS

FIG. 1 shows the enrichment of TIL-B phage displayed Fab libraries for binding of breast cancer cell surface antigens. Libraries were enriched by six sequential rounds of panning and regrowth of cell binding phage. The library derived from patient 14 was panned on MCF7 cells. The patient 16 library was panned on the breast cancer cell lines SKBR3, MCF7, and 2087.

FIG. 2 shows the flow cytometry analysis of phage Fab libraries and individual Fab clones. Libraries 14.6 and 16.6 are shown after enrichment for breast cancer cell binding; individual Fab clones 14.6.11, 14.6.19 (SEQ ID NO: 1; SEQ ID NO: 3 and SEQ ID NO: 4), 14.6.20 (SEQ ID NO: 2; SEQ ID NO: 5 and SEQ ID NO: 6), and 16.4.19) are also shown. Patient 14 library was panned on MCF7 cells; Patient 16 library was panned on MCF7, SKBR3, and 2087 breast cancer cells. Only data from SKBR3 panning is shown.

FIG. 3 shows TILB Fabs are not reactive with actin or p53. Phage displayed Fabs 14.6.11, 14.6.19 (SEQ ID NO: 1; SEQ ID NO: 3 and SEQ ID NO: 4) and 14.6.20 (SEQ ID NO: 2; SEQ ID NO: 5 and SEQ ID NO: 6) were tested for reactivity with actin and p53 by ELISA. Tetnus toxoid antigen and anti-tetnus toxoid phage displayed Fab were included as controls.

FIG. 4 shows expression of soluble Fabs. Fabs 14.6.11, 14.6.19 (SEQ ID NO: 1; SEQ ID NO: 3 and SEQ ID NO: 4), and 14.6.20 (SEQ ID NO: 2; SEQ ID NO: 5 and SEQ ID NO: 6) were expressed as soluble Fabs and purified by His-Tag affinity. Lanes 1, 4, 7, column flow-through; lanes 2, 5, 8 column wash; lanes 3, 6, 9 column elute.

FIG. 5 shows TILB clonality. Clonality was determined by RTPCR and sequencing of IgG heavy chains. Percentages indicate % of total IgG heavy chain sequences for which a clonal relative was identified.

FIG. 6 shows the pattern of clonal expansion of TILBs in infiltrating ductal breast carcinoma. IGHV# indicates germline heavy chain variable gene used by progenitor B cell. Empty circles indicate deduced intermediates; numbers inside circles indicate sequenced clones; numbers next to arrows indicate numbers of mutations in comparison to gernline at a given branching.

FIG. 7 shows a summary of TILB marker expression. Upper table indicates FACS results (underneath table), lower left table indicates results of TILB immunohistochemistry. Numbers in upper table indicate percentage of cells that are positive for the first antigen which are also positive for the second antigen. For example, 95% of CD19+ TIL are also IgG+. These preliminary numbers are based on averages, and have not yet been subjected to statistical analyses.

FIG. 8 shows somatic mutation of IgG heavy chains from TIL-B. Patients=p1, p2, p3; tumor draining lymph node, p3node; healthy donor PBMC, KPBMC.

### DETAILED DESCRIPTION OF THE INVENTION

Unless defined otherwise, all technical and scientific terms used herein have the same meanings as commonly understood by those of ordinary skill in art of the invention. For example, see the definitions provided by U.S. Pat. No 5,955,312 by Hillman and Goli, which is incorporated herein by reference. All publications mentioned herein are incorporated by reference for the purpose of describing and disclosing the cell lines, vectors, and methodologies which might be used in connection with the invention.

Although many different methods and materials similar or equivalent to those described herein can be used in the practice or testing of the present invention, the preferred methods, devices, and material are now described.

It will be appreciated by those skilled in the art that, as a result of the degeneracy of the genetic code, a multitude of Fab-encoding nucleotide sequences, some bearing minimal homology to the nucleotide sequences of any known and naturally occurring gene, may be produced. The invention contemplates every possible variation of nucleotide sequence that could be made by selecting combinations based on possible codon choices. These combinations are made in accordance with the standard triplet genetic code as applied to the nucleotide sequence encoding naturally occurring Fabs, and all such variations are to be considered as being specifically disclosed.

Although nucleotide sequences which encode Fabs and their variants are preferably capable of hybridizing to the nucleotide sequence of the naturally occurring transcription sequences under appropriately selected conditions of stringency, it can be advantageous to produce nucleotide sequences encoding Fabs or their derivatives possessing a substantially different codon usage. For example, codons may be selected to increase the rate at which expression of the peptide occurs in a particular prokaryotic or eukaryotic expression host in accordance with the frequency with which particular codons are utilized by the host. For example, the TAG sequence in Fab 14.6.20 (SEQ ID NO: 2) starting at position 685 encodes a stop codon in mammalian expression systems. Thus, changing that codon to CAG (glutamine) allows full expression to occur withoug affecting function. Other reasons for substantially altering the nucleotide sequence encoding Fabs and their derivatives without altering the encoded amino acid sequences include the production of RNA transcripts having more desirable properties, such as a greater stability or half-life, than transcripts produced from the naturally occurring sequence. Moreover, fragments of the disclosed Fabs may possess moieties that provide breast cancer cell specific binding to take place.

As known by one skilled in the art, a DNA sequence, or portions thereof, encoding Fabs and their derivatives may be produced entirely by synthetic chemistry. Subsequently, the synthetic nucleotide sequence may be inserted into any of the many available DNA vectors and cell systems using reagents that are commonly available. Moreover, synthetic chemistry may be used to introduce mutations into a sequence encoding Fabs or any portion thereof.

Also included within the scope of the invention are polynucleotide sequences that are capable of hybridizing to the nucleotide sequences of SEQ ID NO:1 or SEQ ID NO:2 under various conditions of stringency. Hybridization conditions are based on the melting temperature (Tm) of the nucleic acid binding complex or probe, as taught in Berger and Kimmel (1987, Guide to Molecular Cloning Techniques, Methods in Enzymology, v.152, Academic Press, San Diego, Calif.).

Methods well known in the art can be used to construct expression vectors containing sequences encoding a Fab and appropriate transcriptional and translational control elements. Methods may include in vitro recombinant DNA techniques, synthetic techniques, and in vivo genetic recombination in a variety of expression vector/host systems, such as bacteria transformed with recombinant bacteriophage or plasmids or insect cell systems infected with viral expression vectors such as the baculovirus. These methods are described in standard laboratory references, such as Sambrook, J. et al. Molecular Cloning, A Laboratory Manual, Cold Spring Harbor Press, Plainview, N.Y. (1989).

Altered nucleic acids encoding Fabs which may be used in accordance with the invention include deletions, insertions or substitutions of different nucleotides resulting in a polynucleotide and polypeptide that encodes the same or a functionally equivalent Fab. The protein may also show deletions, insertions or substitutions of amino acid residues which produce a silent change and result in functionally equivalent Fab. Deliberate amino acid substitutions may be made on the basis of similarity in polarity, charge, solubility, hydrophobicity, hydrophilicity, and/or the amphipathic nature of the residues as long as the biological activity of Fab is retained. For example, negatively charged amino acids aspartic acid and glutamic acid might be substituted for one another.

Also included within the scope of the invention are alleles encoding Fab. As used herein, an “allele” or “allelic sequence” is an alternative form of the nucleic acid sequence encoding Fab. Alleles result from a mutation, i.e. a change in the nucleic acid sequence, and generally produce altered mRNAs or polypeptides whose structure or function may or may not be altered. Any given gene may have none, one or many allelic forms. Common mutational changes which give rise to natural deletions, additions or substitutions of amino acids. Each of these types of changes may occur alone, or in combination with the others, one or more times in a given sequence. For example, the TAG sequence in Fab 14.6.20 (SEQ ID NO: 2) starting at position 685 may be changed to TCG (serine) to reflect an allele found in parent B cells.

Many ways exist in the art by which Fab may be used therapeutically. Examples include, but are not limited to, administering Fab through the introduction of an expression vector into a subject for in vivo therapy or administering Fab as part of a pharmaceutical composition. Depending on the route of administration, appropriate agents for use in combination with Fab for therapy may include any conventional pharmaceutical carrier such as saline or buffered saline (intravenous dosing) and dextrose or water (oral dosing). Further details on techniques for formulation and administration may be found in the latest edition of Remington's Pharmaceutical Sciences (Maack Publishing Co., Easton, Pa.).

Breast tumor-infiltrating B cells (TIL-B) in tumors derived from geriatric patients have been investigated with the goal of developing new diagnostic or therapeutic antibodies. In discovering the present invention, the histology, phenotype, IgG repertoire and immunoglobulin specificity of tumor infiltrating B cells (TIL-B) from infiltrating ductal carcinomas of the breast were examined. Patient data and work done are summarized below in table I:

TABLE 1
Summary of patient data and work done in preliminary study. Patients
were designated numerically in order of collection; Phage display indicates
Fab library cloned, GC IHC indicates germinal center immunohistochemistry
performed; LN IHC indicated lymph node immunohistochemistry performed;
TIL FACS indicates flow cytometry analysis of TIL performed; grade indicates
tumor grade; ER/PR indicates % cells positive for estrogen or progesterone
receptor; over 2+ indicates HER2+; MIB1 proliferation index; IDC , infiltrating
ductal carcinoma; ILC , infiltrating lobular carcinoma.

| patient | phage display | GC IHC | LN IHC | TIL FACS | Age | Grade | ER/PR | Her2 | MIB-1 | Diagnosis |
|---|---|---|---|---|---|---|---|---|---|---|
| 3 | | | x | | | | | | | |
| 5 | | | x | | 61 | 1 | 95/<2 | N | 2% | IDC |
| 8 | | x | | x | 83 | 2 | 99/65 | 1+ | 9% | IDC |
| 10 | | | x | | | | | | | |
| 12 | | x | | | 39 | 3 | N/N | N | 75% | IDC |
| 13 | | x | | | 77 | 2 | N/90% | N | <5% | mixed |
| 14 | x | x | | | 78 | R2 | 50/N | RN | <2% | IDC |
| 16 | x | x | | | 73 | 3 | N/N | 2+ | 60% | IDC |
| 25 | x | x | | | 61 | 2 | 100/86 | 1+ | 31% | IDC/ILC |
| 26 | x | x | | | 48 | 3 | N/N | N | >95% | IDC |
| 30 | | x | | | 54 | 3 | N/N | N | 64% | IDC |
| 32 | | x | | | 70 | 2, 3 | N/N | 2+ | 26% | IDC |
| 33 | | | | x | 79 | 2 | 95/90 | N | 6% | IDC |
| 35 | | | | x | 58 | 3 | N/N | 3+ 100% | 12% | IDC |
| 36 | | | | x | 58 | 3 | >95/N | 3+ | 39% | IDC |
| 39 | | | x | x | 65 | 3 | N/<2% | 3+ | 52% | IDC |
| 40 | | | x | x | 45 | 3 | 100/N | 1+ | 30% | IDC |

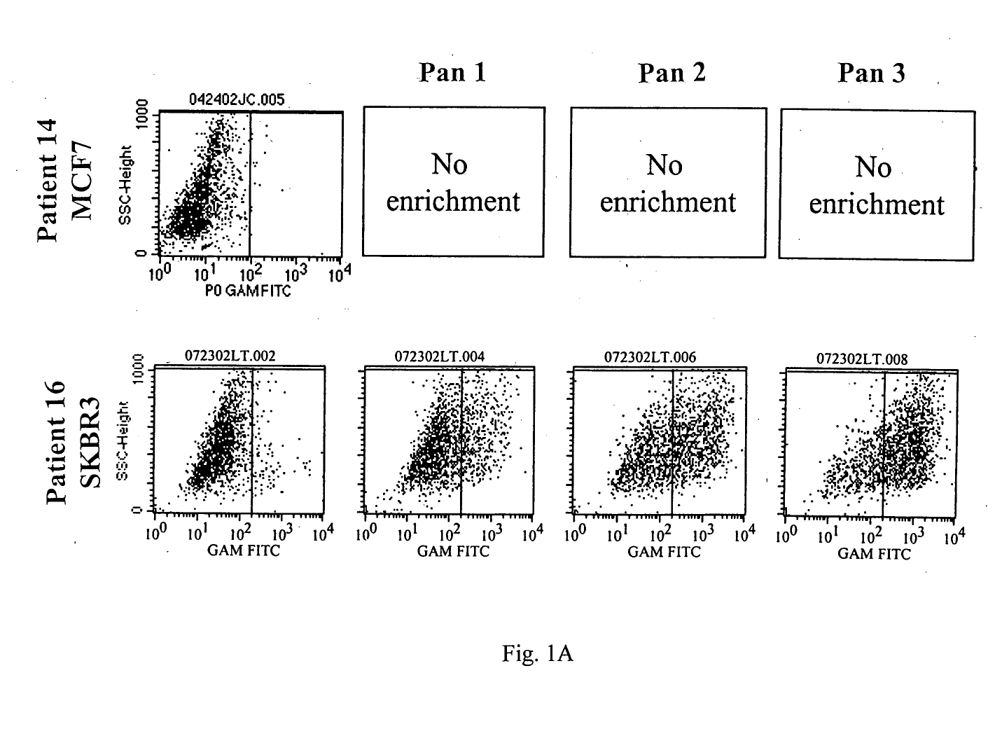

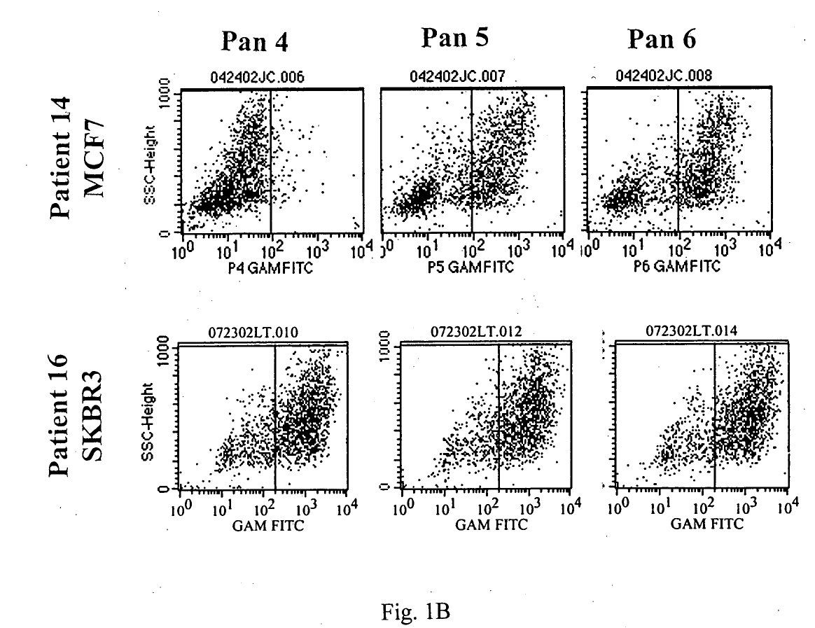

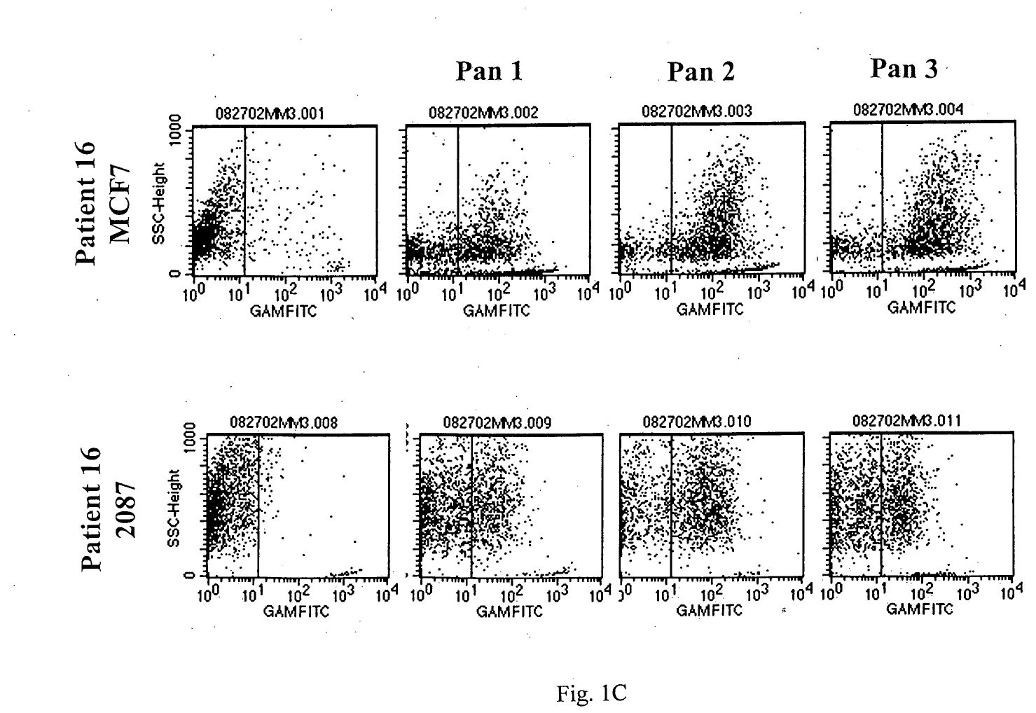

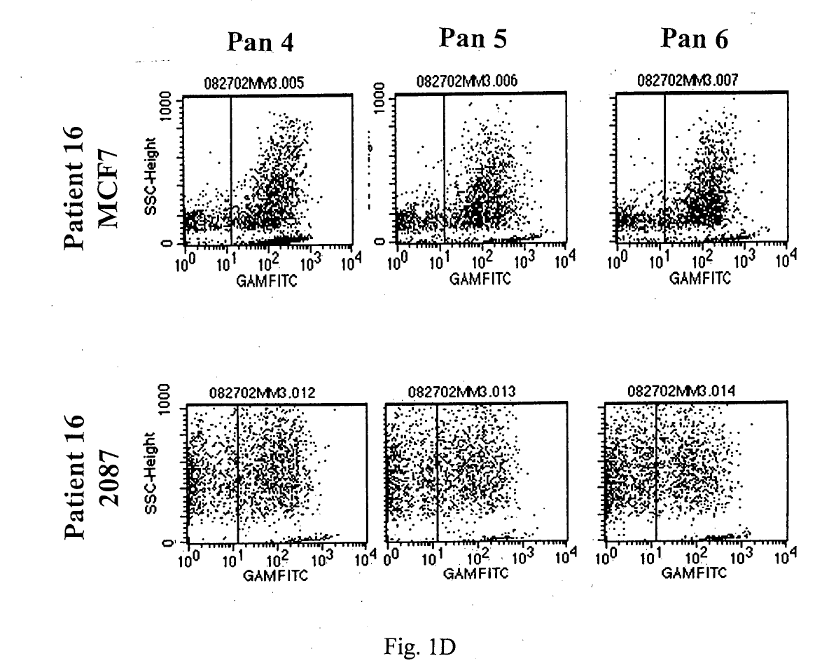

To determine if TIL-B immunoglobulins were reactive with tumor, 2 phage-displayed immunoglobulin Fab libraries were generated from geriatric patient TILs by the methods of Barbas et al (Barbas, C. F. 2000. Phage display: a laboratory manual. Cold Spring Harbor Labortory Press, Cold Spring Harbor, N.Y.). Fab (heavy chain variable region plus CH1 and light chain) libraries of ˜1×10⁷ Fab clones were cloned in the pCOMBX phage display vector (gift of C. Barbas, Scripps Research Institute, San Diego, Calif.). The inventor Julia Coronella received training in phage display from Dr. Carlos Barbas of Scripps Research Institute at the Cold Spring Harbor course on phage display 2000. The libraries were panned on cultured breast cancer cells in order to enrich for Fabs that bind breast cancer cell surface antigens. Panning consisted of 6 sequential rounds of incubation of phage displayed Fabs with breast tumor cells under increasingly stringent conditions to isolate high affinity Fabs, and regrowth of the binding fraction. Enrichment of patient 14 Fab library for tumor cell binding was observed after 4 sequential rounds of panning on MCF7 cells (FIG. 1). Enrichment of patient 16 Fab library for tumor cell binding was observed after only 1 panning on SKBR3, MCF7 and 2087 breast cancer cells, indicating a high fraction of tumor binding Fabs in the library (FIG. 1).

Individual Fab clones were selected from the enriched libraries and assessed for tumor cell surface binding by flow cytometry. Fabs were selected from all panning passages showing enrichment. Clones with identical sequences were not multiply analyzed. Clones with low or absent tumor cell binding were discarded. Because of the inherent variability in phage Fab binding assays (phage Fab are unstable and must be grown and prepared prior to each new experiment), all flow cytometry and ELISA assays were replicated multiple times. Flow cytometry data are summarized in table 2 below:

TABLE 2
Flow cytometry analysis of phage display Fab libraries and individual Fabs. Patient 14
library data is shown for MCF7 panning; patient 16 library data is shown for SKBR3 panning.

| | MCF7 | SKBR3 | 3133 | 3199 | 2087 | HMEC | A549 | HL60 | FF | B16NEO | B16MUC1 | C57MUC1 |
|---|---|---|---|---|---|---|---|---|---|---|---|---|
| antibody | | | | | | | | | | | | |
| 14.6 | hi | med | lo | hi | med | 0 | med | 0 | 0 | lo | lo | 0 |
| 14.6.11 | hi | hi | lo | hi | med | 0 | 0 | 0 | 0 | 0 | 0 | 0 |
| 14.6.19 | hi | 0 | lo | lo | 0 | 0 | 0 | 0 | 0 | 0 | 0 | 0 |
| 14.6.20 | hi | hi | lo | med | med | 0 | 0 | 0 | 0 | 0 | 0 | 0 |
| 16.60 | hi | hi | | | | | hi | 0 | | | | |
| 16.4.19 | hi | hi | | | | | 0 | 0 | | | | |
| antigen | | | | | | | | | | | | |
| HER2 | 0 | hi | med | 0 | med | | 0 | | | | | |
| MUC1 | lo | 0 | med | 0 | lo | | 0 | | | 0 | hi | hi |
| CEA | 0 | 0 | lo | 0 | 0 | | med | | | | | |
| EpCAM | 0 | | hi | hi | 0 | | | | | | | |

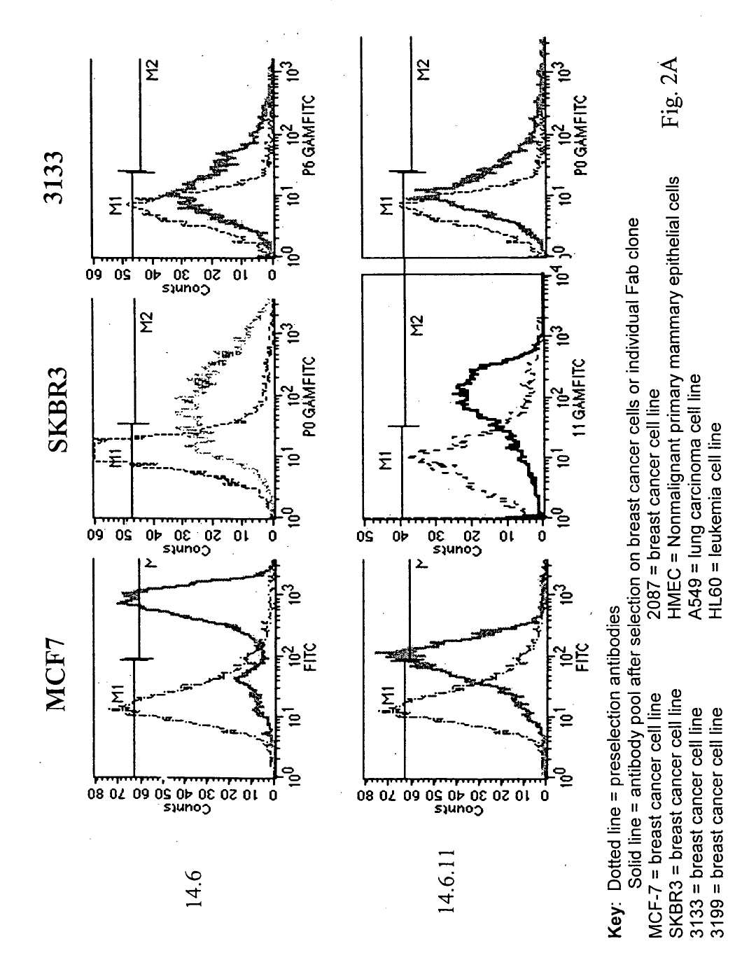

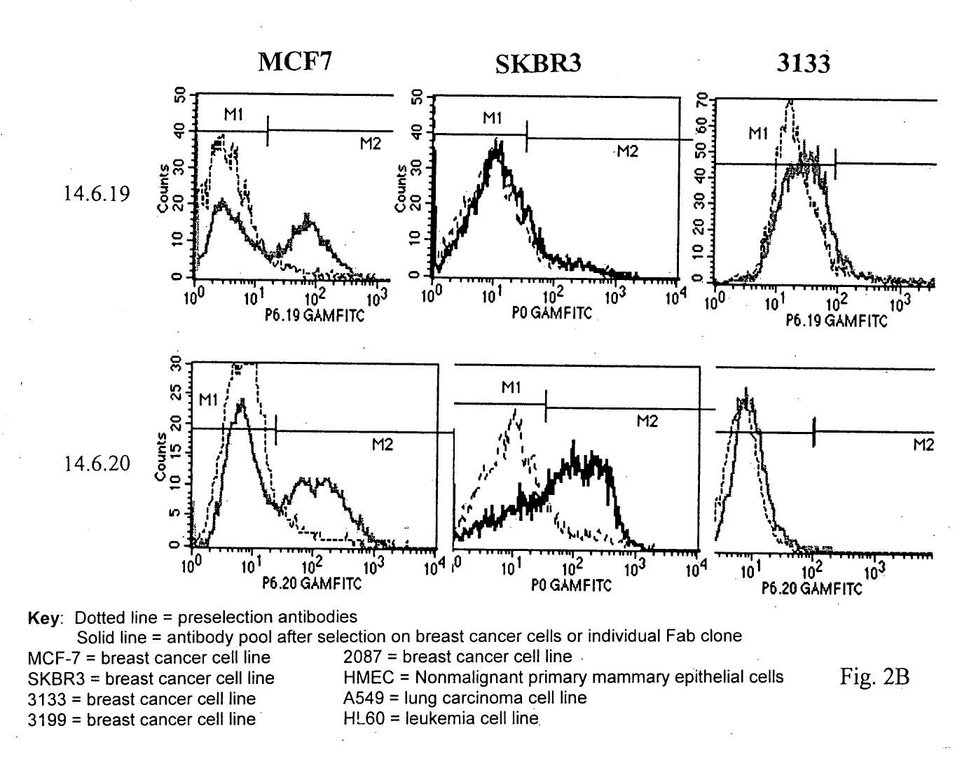

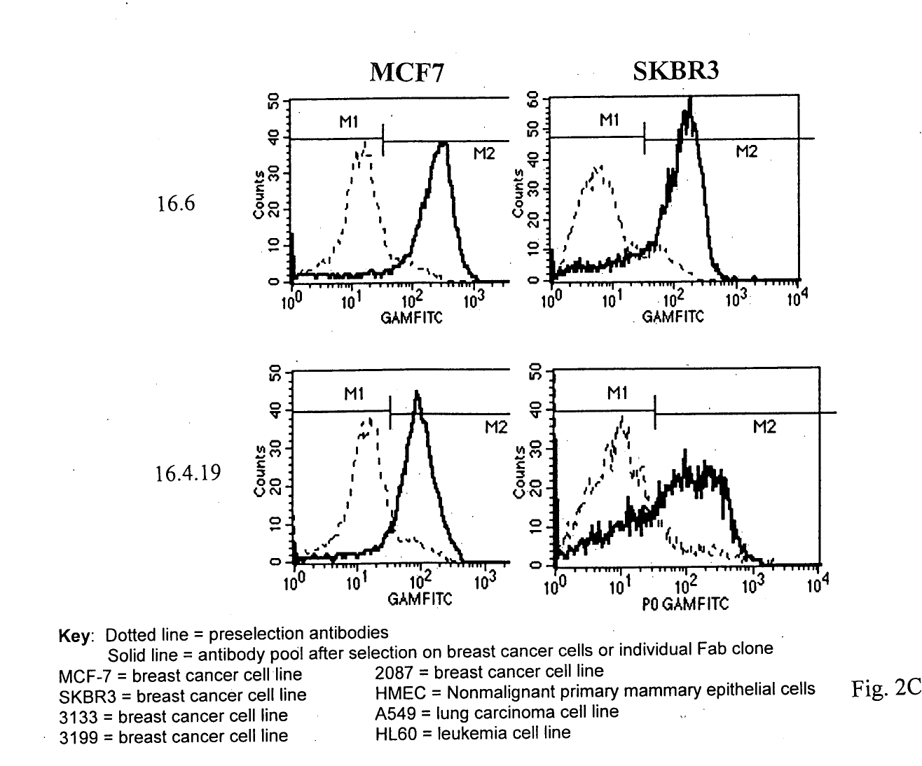

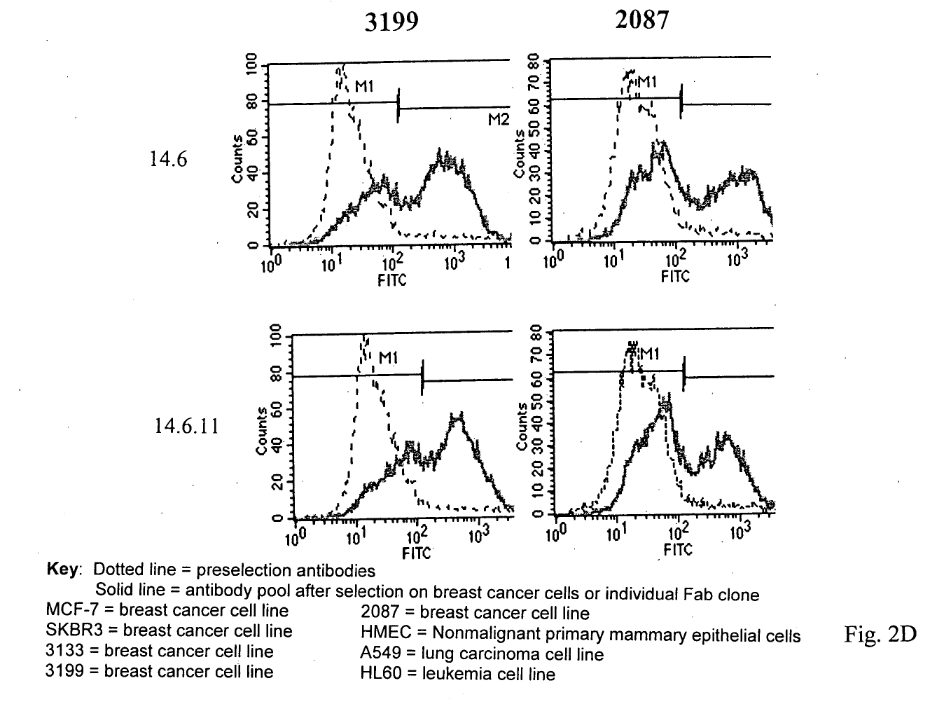

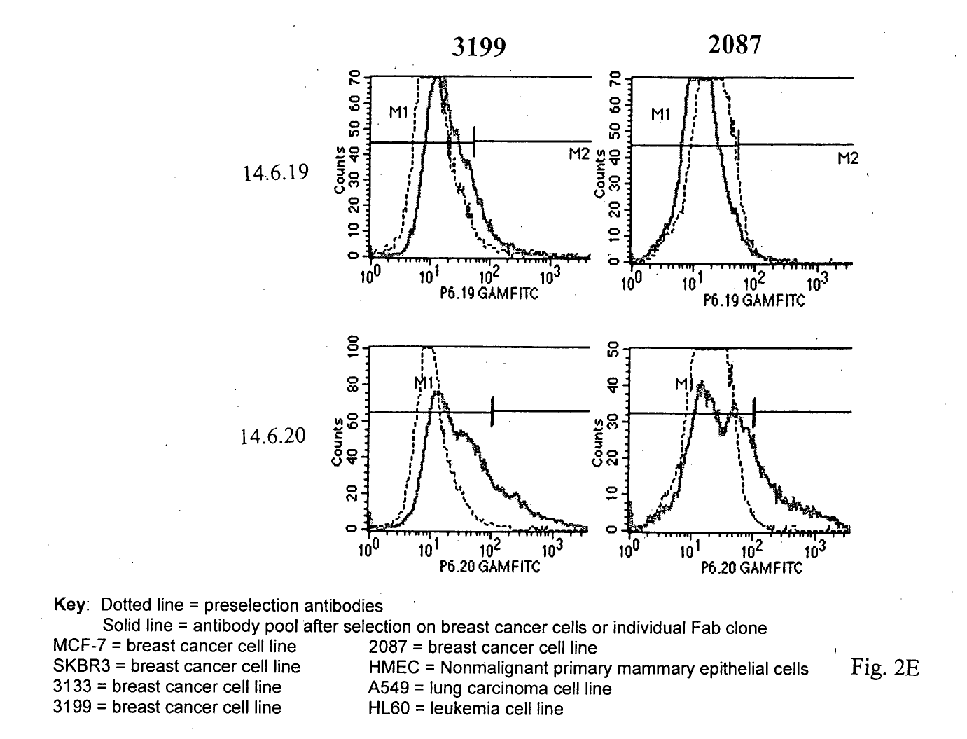

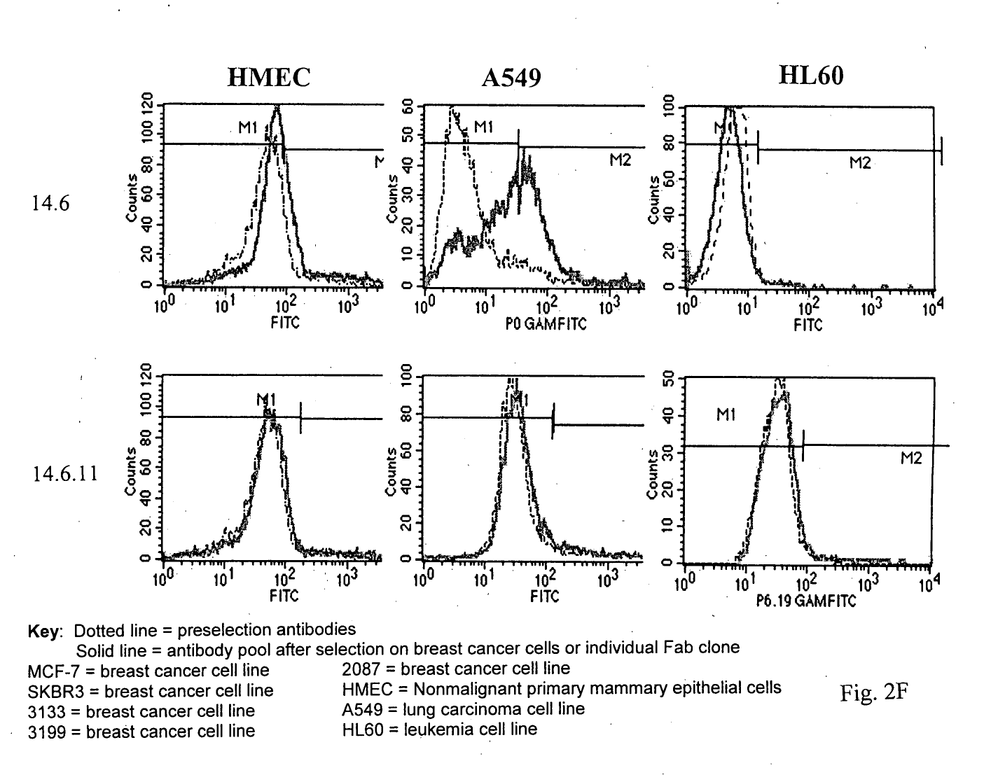

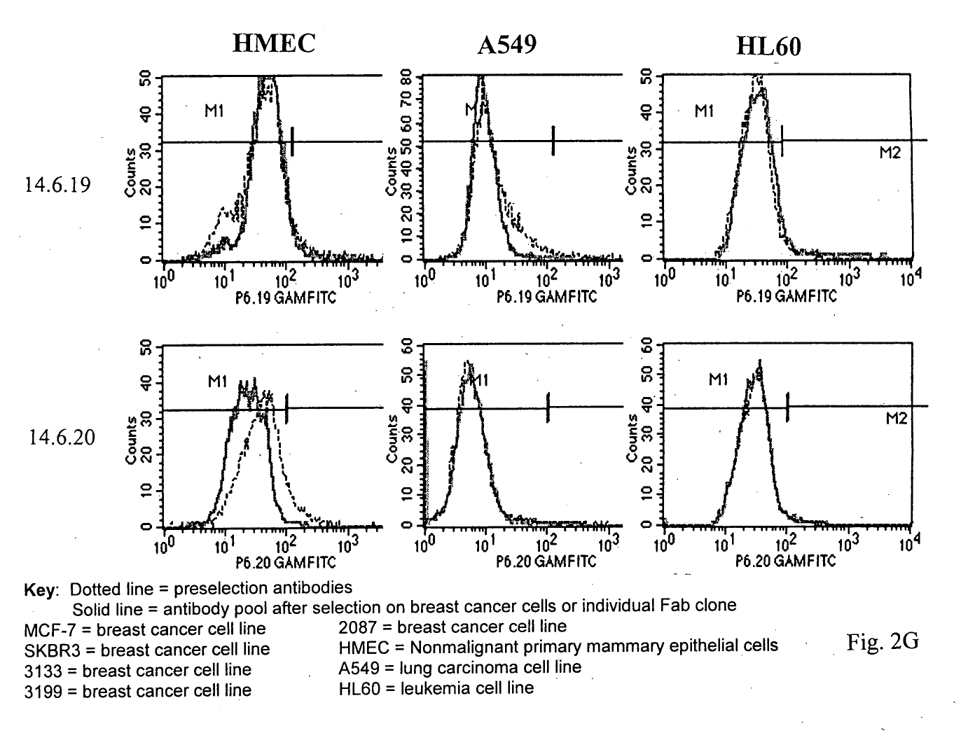

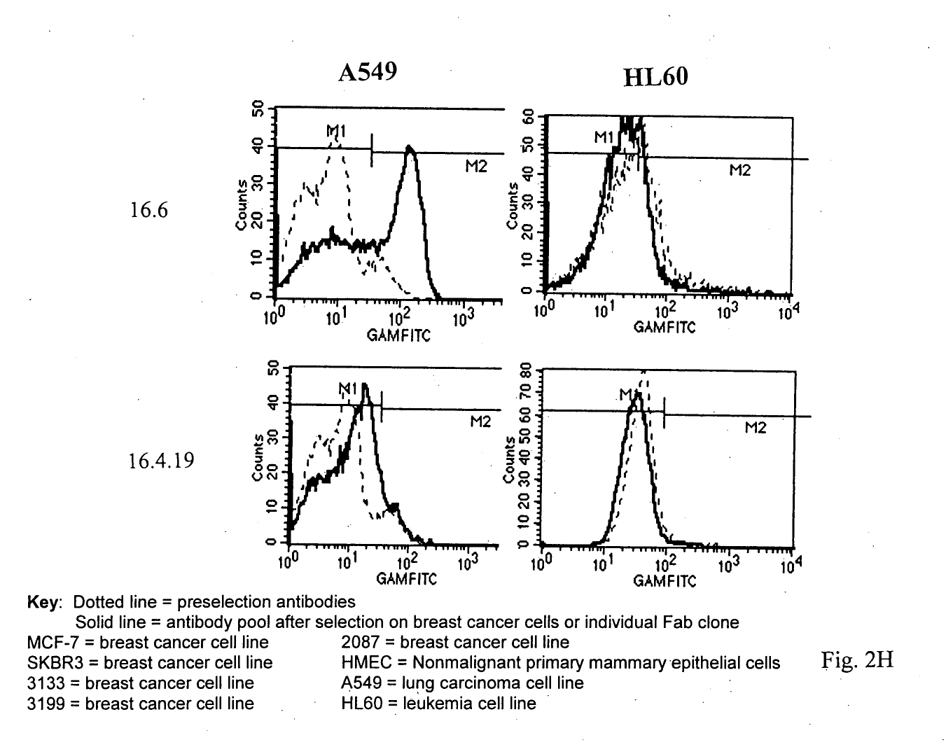

Thus far, 3 Fabs with apparent specificity for breast tumor cells have been isolated from the patient 14 Fab library (FIG. 2). Fab 14.6.11 binds all breast cancer cell lines tested but not nonmalignant healthy breast epithelium, primary fibroblasts or the leukemia cell line HL60. Fabs 14.6.19 (SEQ ID NO: 1; SEQ ID NO: 3 and SEQ ID NO: 4) and 14.6.20 (SEQ ID NO: 2, SEQ ID NO: 5 and SEQ ID NO: 6) were also highly specific for breast cancer, but less universal binding of breast cancer cell lines was observed. A number of promising Fabs were isolated from the patient 16 library and are currently under analysis. Monovalent phage Fabs have low avidity, and the FACS protocol utilized is a lengthy multi-step staining. Therefore, Fab clones that exhibit binding are likely of high affinity, with low off-rates. The 3 Fabs contain somatic mutations in the antigen-binding CDR regions, suggesting affinity maturation.

As the first stage of antigen identification, Her-2/neu, MUC1, CEA, EpCAM, B-actin and p53 were eliminated as possible antigens. While it is not possible to test all known breast cancer antigens prior to proceeding to antigen identification, it is desirable to eliminate the most obvious. Binding of the Fab clones 14.6.11, 14.6.19 (SEQ ID NO: 1; SEQ ID NO: 3 and SEQ ID NO: 4) and 14.6.20 (SEQ ID NO: 2; SEQ ID NO: 5 and SEQ ID NO: 6) to cell lines was compared to expression levels of antigens on these cell lines, summarized in table 2 (above).

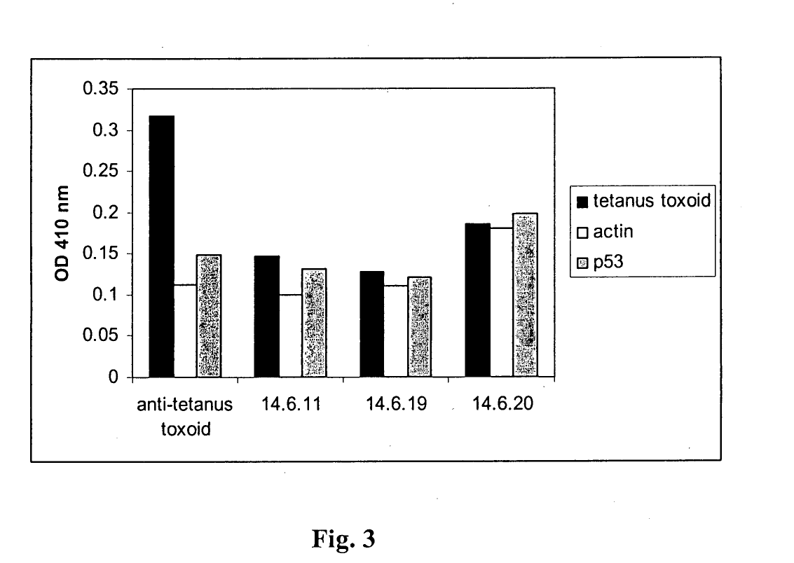

These data suggests that Her-2/neu, MUC1, CEA and EpCAM are not the antigens with which any of these Fabs react. The lack of MUC1 binding was confirmed by FACS with MUC1-transfected cell lines (gift of S. Gendler, Mayo Clinic, Scottsdale, Ariz.). Fabs were also assayed for binding of B-actin and p53 by ELISA. No binding was observed (FIG. 3). Fab clones 14.6.11, 14.6.19 (SEQ ID NO: 1; SEQ ID NO: 3 and SEQ ID NO: 4) and 14.6.20 (SEQ ID NO: 2; SEQ ID NO: 5 and SEQ ID NO: 6) utilize unique germline immunoglobulin genes (table 3, below), and have non-identical patterns of cell line binding, consistent with binding of unique antigens.

TABLE 3
Patient 14 Fab germline genes.

| | Heavy Chain | | | Light Chain | |
|---|---|---|---|---|---|
| Fab | V | D | J | V | J |
| 14.6.11 | IGHV4-59*01 | IGHD3-10*01 | IGHJ5*02 | IGKV1-5*03 | IGKJ1*01 |
| 14.6.19 | IGHV3-74*01 | IGHD3-16*01 | IGHJ4*02 | IGKV2D-28*01 | IGKJ5*01 |
| 14.6.20 | IGHV3-23*01 | IGHD3-3*01 | IGHJ4*02 | IGKV1-5*03 | IGKJ4*01 |

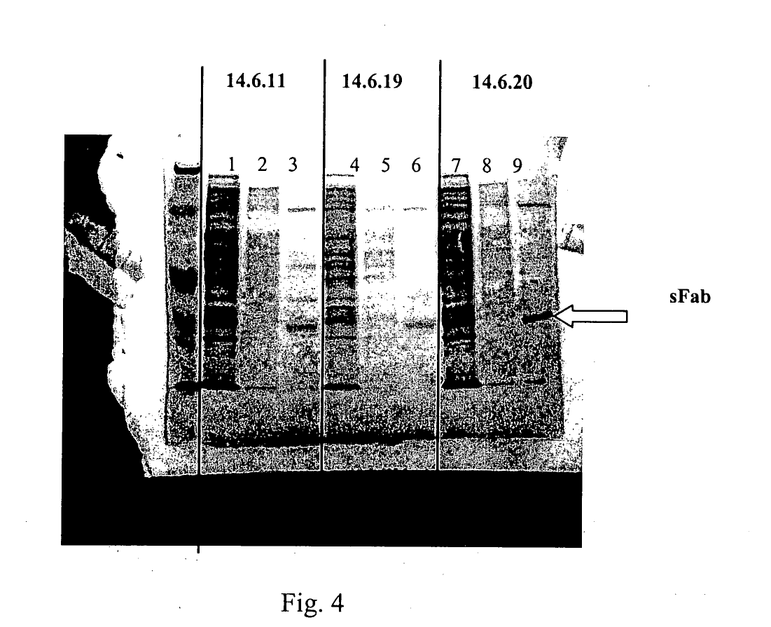

One strategy to identify the breast cancer cell surface antigens that are reactive with the cloned Fabs is immunoprecipitation followed by mass spectrometry. To this end, Fab clones 14.6.11, 14.6.19 and 14.6.20 were transformed into the non-supressor cell line Top10′F (Invitrogen) to allow expression of soluble Fabs. The pCombX phage display vector contains an amber codon between the Fab and gene III capsid protein to allow expression of Fabs without gene m fusion and display on the capsid. Fab clones 14.6.11, 14.6.19 (SEQ ID NO: 3 and SEQ ID NO: 4) and 14.6.20 (SEQ ID NO: 5 and SEQ ID NO: 6) were expressed and purified by His-tag affinity (FIG. 4). Following purification, Fabs were linked to protein A-agarose. Cell membrane proteins were solubilized from the breast cancer cell lines MCF7 and 3199 for immunoprecipitation with the Fabs. Immunoprecipitations are in progress, and will be analyzed by SDS-PAGE, followed by mass spectrometry (a service of the AZCC core proteomics facility).

Unlike T cells, the anti-tumor activity of B cells normally occurs in a lymph node (activation/affinity maturation/differentiation/antibody production) or bone marrow (antibody production) rather than at the site of antigen in the periphery (with the exception of IgA-producing plasma cells at the mucosa, which produce low-affinity antimicrobial “natural antibodies”). With the exception of autoimmune diseases, no precedent exists for local B cell-mediated immune reactions in peripheral tissues. TIL-B were studied by immunohistochemistry, flow cytometry, and immunoglobulin sequencing in order to understand the interaction between tumor and B lymphocytes in the tumor microenvironment.

To determine if the aggregates of B cells observed in tumors were the result of random recruitment from the periphery or the proliferation of tumor-infiltrating lymphocytes, IgG heavy chain libraries were generated by RTPCR and random clones sequenced from a total of six breast tumors, a tumor-draining lymph node, and the peripheral blood of a healthy donor. Between 12 and 58 IgG heavy chains were sequenced for each sample.

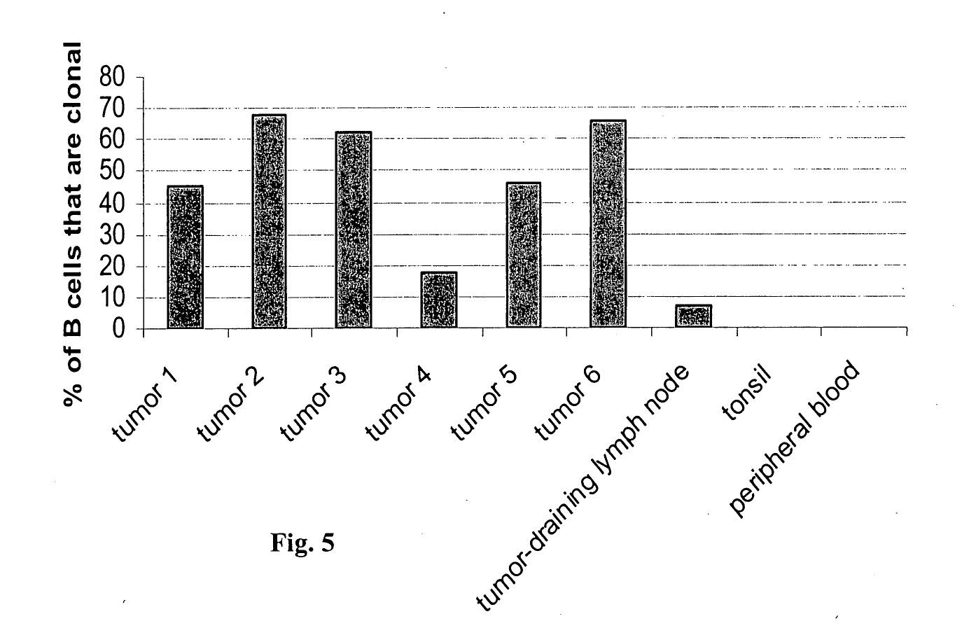

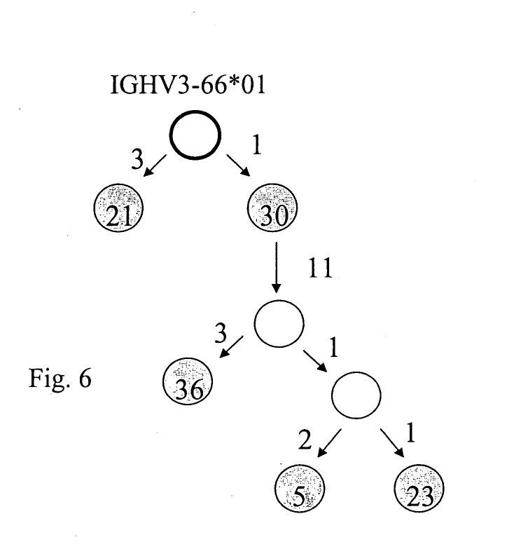

Peripheral blood lymphocytes were included in order to control for poor PCR methodology that could result in repetitive cloning of single PCR products rather than as a measure of the peripheral repertoire, which has been extensively characterized elsewhere. The inventor's peripheral repertoire results are equivalent to those published previously. B cell clones occur very infrequently (<1/20,000) in the peripheral blood of both young and elderly humans, and are not detectable by sampling and sequencing methods such as those employed in this study. Oligoclonal expansion of TIL-B was established by the presence of intratumoral clonal groups derived from common progenitor B cells in all tumors examined (FIG. 5). Between 18 and 68% of IgG heavy chain sequences from TIL-B belonged to clonal groups, while only 7% of tumor-draining lymph node sequences and 0% of peripheral blood sequences were clonal, consistent with the expected large repertoires of those populations. As occurs in lymph node germinal centers, TIL-B lineages accumulated unique somatic mutations during proliferation, allowing the derivation of genealogical trees and calculation of cell doubling numbers (FIG. 6). For example, clone 36 in FIG. 6 contains 14 unique mutations in the 296 base pair VH region in comparison with clone 30, a linear predecessor in this TIL-B lineage. Based on a somatic hypermutation level of 1 base pair per 10² to 10³ bases per generation for immunoglobulins, between 4.7 and 47 cell divisions would be required.

Based on immunohistochemistry, it was determined that approximately 20% of breast tumors contain significant numbers of B cells, in agreement with previously published percentages. Seven tumors containing TIL-B were selected for further immunohistochemistry. Although tumor-infiltrating lymphocytes were found scattered throughout the stroma and interspersed between tumor cells in all tumors, CD20+ B lymphocytes occurred exclusively in dense aggregates. In most cases, TILB aggregates occurred in stromal areas immediately adjoining tumor nests, and were not observed outside the tumor margins. All TILB aggregates contained interdigitating CD21+ follicular dendritic cells. CD21+ cells were not observed outside of B cell germinal centers. Germinal centers were surrounded by CD3+ T lymphocytes, the majority of which were CD4+, although a component of CD8+ cells were also present. Plasma cells (CD38) and NK cells (CD56) were rare, and occurred randomly in relation to other lymphocytes (data not shown). Most B cells were Ki-67−, indicating that the clonal groups observed in immunoglobulin sequencing were the result of slow or previous proliferation. Most germinal center B cells were positive for BCL2 and HLA-DR, but negative for CD10, CD27, and CD38, suggesting an activated but not memory or plasma cell phenotype.

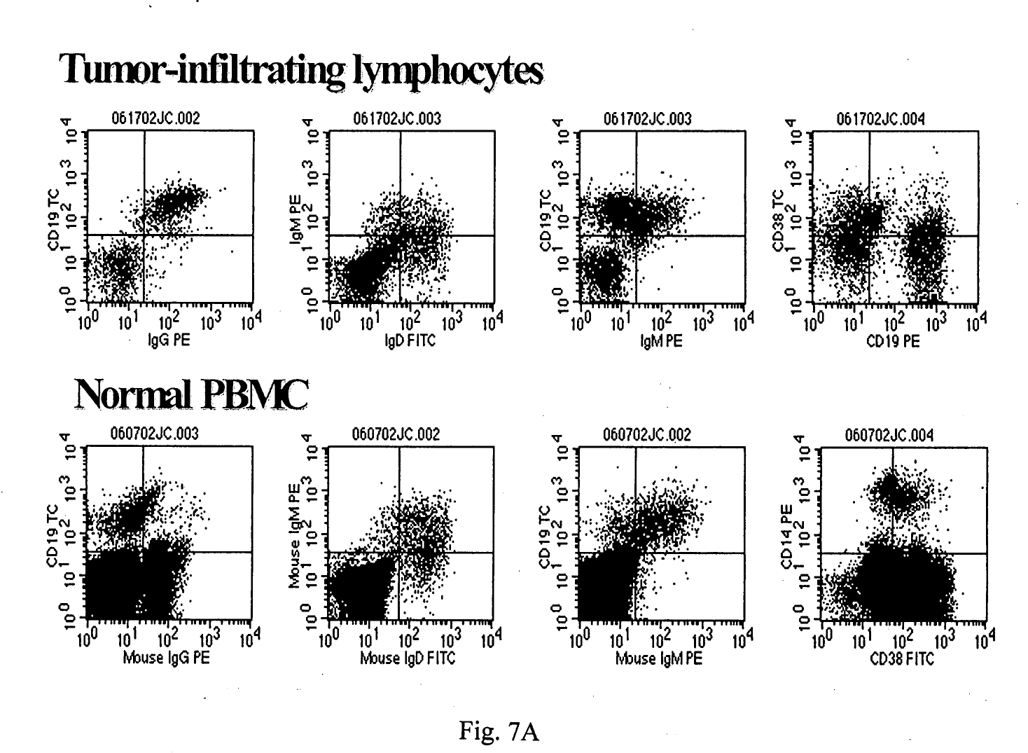

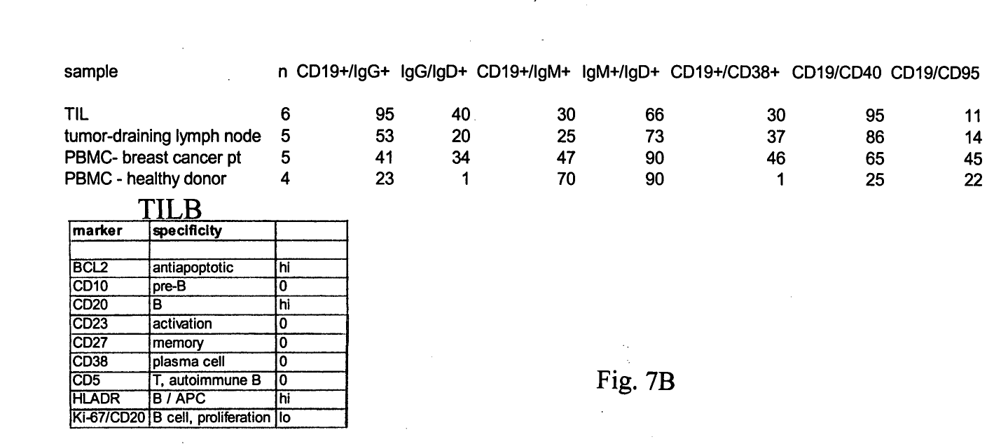

In addition, flow cytometry was performed to assess the presence of IgG, IgM, IgD, CD38, CD5 (associated with autoimmunity), CD95, and CD40 on CD19+ B cells. Although CD38 was not detected by immunohistochemistry, flow cytometry is more sensitive and can detect CD381o cells. Six breast tumors, 5 tumor-draining lymph nodes, peripheral blood from 5 breast cancer patients, and peripheral blood from 4 healthy donors were analyzed. Data is summarized in FIG. 7. As occurs in a lymph node follicle, the populations of TILB were heterogeneous; Most TILB were IgG+, IgM−, and approximately half were IgD+. All TILB were CD5−. Some B cells expressed low levels of CD38, as do centroblasts and centrocytes. However, no CD38hi cells were observed, consistent with the lack of plasma cells determined by immunohistochemistry. The absence of plasma cells can be explained by apoptosis or alternatively, by a deficit in the plasma cell differentiation pathway. Centroblasts and centrocytes are normally CD95+, and very sensitive to apoptosis. In contrast, most TILB express CD40, but not CD95 (Fas). Strong BCL2 expression of TILB was determined by immunohistochemistry, and is unusual in that only plasma cells and germinal center founder cells normally express this antiapoptotic protein. BCL2 expression protects germinal center B cells from apoptosis in vivo. Through expression of the antiapoptotic proteins CD40 and BCL2, and lack of CD95 expression, TILB may be unusually resistant to apoptosis. Resistance to apoptosis is consistent with the inventor's demonstration of autoreactive antibodies from TILB, as autoreactive B cells are normally deleted during the germinal center reaction. One can speculate that cytokines from local activated T lymphocytes or perhaps cytokine secretion from the tumor itself may induce this antiapoptotic profile.

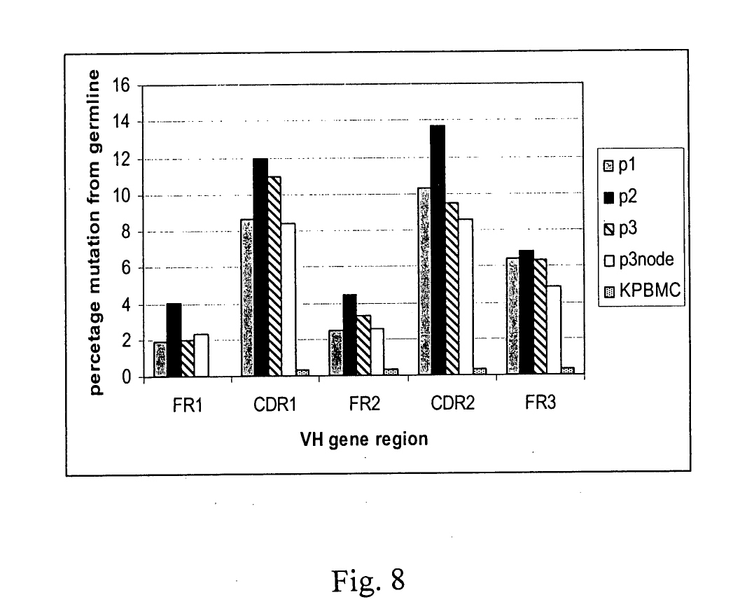

TIL-B IgG heavy chain mutation levels, patterns and germline gene usage suggest that TIL-B undergo affinity maturation intratumorally, presenting the possibility of production of high-affinity anti-tumor immunoglobulin. However, this conclusion stems from indirect evidence of affinity maturation, which can only be resolved through antigen affinity studies. TIL-B IgG heavy chains contained somatic mutations that clustered in the antigen-contacting CDR regions, as previously observed in affinity-matured antibodies, and as was also seen in tumor-draining lymph node but not peripheral blood IgG (FIG. 8). Although the peripheral blood control was from a younger individual (39 years of age), previous studies demonstrate that overall levels of VH hypermutation in peripheral blood is equivalent in young and elderly adults. As calculated by the polynomial algorithm of Lossos et al., replacement and silent mutations occurred nonrandomly in 36-84% of TIL-derived IgG heavy chains. Further, low levels of TIL-B IgG heavy chain nonsense mutation and a modest bias in germline gene usage suggested clonal selection.

In order to further illustrate the invention, the following example is provided. While this example is contemplated to be the preferred mode, it will be understood by those in the art that numerous alternative methodologies may be successfully practiced in lieu of the preferred method described herein. Therefore, this example is not intended in any way to limit the invention.

### EXAMPLE

Experimental Procedures for Fabs 14.6.19, and 14.6.20. Cloning history: A Fab library was cloned from breast tumor-infiltrating B cells by RTPCR, as published in Coronella, J. A. et al., 2002. Antigen-driven oligoclonal expansion of tumor-infiltrating B cells in infiltrating ductal carcinoma of the breast. J. Immunology 169:1829.

The library was subcloned into the pCOMBX phage display vector (gift of C. Barbas, Scripps Research Institute, La Jolla, Calif.). Fabs were isolated from the library on the basis of cell-surface reactivity with MCF7 cells. Two Fabs so isolated were 14.6.19, and 14.6.20, the nucleotide and peptide sequences of which are hereinafter described.

[image]

[image]

[image]

The Fabs were subsequently sent to IDEC Pharmaceuticals, and subcloned into the N5 mKm vector (property of IDEC). A change was made to the 14.6.20 Fab, mutating the TAG amber codon in the VH region (in white text above) to CAG, encoding Gln.

Flow cytometry analysis of Fabs: below are the binding profiles of the two Fabs with a number of cancer and non-cancer cell lines.

| | MCF7 | SKBR3 | 2087 | 3133 | 3199 | MDA | HMEC |
|---|---|---|---|---|---|---|---|
| | Breast cancer | Breast cancer | Breast cancer | Breast cancer | Breast cancer | Breast cancer | primary |
| | | | | | | | breast epithelium |
| 14.6.19 | + | + | − | + | + | | − |
| 14.6.20 | + | + | + | − | + | + | − |
| | B16NEO | B16MUC1 | C57MUC1 | FF | | HeLa | PANC-1 |
| | murine | murine + MUC1 | murine + MUC1 | primary foreskin fibroblasts | | cervical ca | pancreatic ca |
| 14.6.19 | − | − | − | − | | − | |
| 14.6.20 | − | − | − | − | | + | − |
| | A549 | HL60 | U251 | SW480 | JORP | DU-145 | OVCAR |
| | lung cancer | leukemia | glioma | colon ca | melanoma ca | prostate ca | ovarian ca |
| 14.6.19 | + | − | − | + | + | + | + |
| 14.6.20 | − | − | − | − | + | + | +/− |

No information exists regarding the 14.6.19 antigen. The 14.6.20 antigen is resistant to trypsin and glycopeptidase F, but partially sensitive to periodate treatment, suggesting a protein epitope on a cell surface glycoprotein. Based on preliminary Western blot analysis, the antigen may be ˜129 kDa.

Although the invention has been described with reference to various applications, methods, and compositions, it will be appreciated that various changes and modifications may be made without departing from the invention. The foregoing examples are provided to better illustrate the invention and are not intended to limit the scope of the invention.

What is claimed is:

1. An isolated polynucleotide encoding a breast cancer-specific antibody fragment including SEQ ID NO:1.

2. A hybridization probe comprising the polynucleotide of claim 1 and a detectable label.

3. A polynucleotide fragment that is fully complementary to a corresponding segment of the polynucleotide of claim 1.

4. An isolated polynucleotide encoding a breast cancer-specific antibody fragment including SEQ ID NO:2.

5. A hybridization probe comprising the polynucleotide of claim 4 and a detectable label.

6. A polynucleotide fragment that is fully complementary to a corresponding segment of the polynucleotide of claim 4.

7. An isolated antibody or antibody fragment that binds to breast cancer cells and contains the amino acid sequence of SEQ ID NO:3.

8. An isolated antibody or antibody fragment that binds to breast cancer cells and contains the amino acid sequence of SEQ ID NO:4.

9. An isolated antibody or antibody fragment that binds to breast cancer cells and contains the amino acid sequence of SEQ ID NO: 5.

10. An isolated antibody or antibody fragment that binds to breast cancer cells and contains the amino acid sequence of SEQ ID NO: 6.

11. A method for screening breast cancer cells, comprising the step of contacting said breast cancer cells with one or more antibodies that contain one or more sequences selected from the group consisting of SEQ ID NO: 3, SEQ ID NO: 4, SEQ ID NO: 5, or SEQ ID NO: 6.


SEQUENCE LISTING (6 sequences)

SEQ ID NO: 1 - DNA, 1405 bp, Homo sapiens
aaaatgcctggctggtttcgctaccgtggcccaggcggccgagctcgtgatgactcagtctccactctccctgcccgtcacccctggagagccggcctccatctcctgcaggtctagtcagagtctcctgcatagtaatggatacaactatttggattggtacctgcagaagccagggcagtctccacagctcctgatctatttgggttttaatcgggcctccggggtccctgacaggttcagtggcagtggatcaggcacagattatacactgaaaatcagcagagtggaggctgaggatgttggggtttattactgcatgcaaggtctacaaactcctaggaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttagttctagataattaattaggaggaatttaaaatgaaatacctattgcctacggcagccgctggattgttattactcgctgcccaaccagccatggcccaggtgcagctgcaggagtccgggggaggcttagttcagcctggggggtccctgagactctcctgtgaagcctctggatacaccttcagcaattactggatgcactgggtccgccaacctccagggaaggggctggtgtgggtctcacgtattaatgaagatgggagtatcacaaacgacgcggactccgtgaagggccgatccaccatctccagagacaacgccaagaacacgctgtatctggaaatgaacagtctgagagccgaggacacggctgtctattactgtacacgagatattgggggtcgtgatgctcactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaat

SEQ ID NO: 2 - DNA, 1424 bp, Homo sapiens
actggctggtttcctaccgtggcccaggcggccgagctccagatgacccagtctccttccaccctgtctgcatctgtaggagacagagtcaccatcacttgccgggccagtcacagtgttagtgggtggttggcctggtatcagcagaaaccagggaaagcccctaagctcctgtcctatgaaccgtctagtttggaaagtggggtcccatcaaggttcagcggcagtggatctgggacagaattcactctcaccatcagcagtctgcaacctgaagattttgcaacttactactgtcaagagagttaccgtatcacttccctcactttcggcggagggaccaaggtggagaccagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttagttctagataattaattaggaggaatttaaaatgaaatacctattgcctacggcagccgctggattgttattactcgctgcccaaccagccatggccgaggtgcagctggtgcagtctgggggaggcttagttcagcctggggggtccctgagactctcctgtacagcctctggattcatctttaataactatgccatgtcctgggtccgccaggctccagggaagggcctagaatgggtctcaggtattagtactggtggtagcagcacataccacgcggactccgtgaagggccggtttaccatctccagggacaatttcaagaagacactgtggctacaaatgaacagcctgacaccagaggacgcggccgtctactactgtgcgagacatgcgaatttttggaatggttatttgtaggaaaagggggcgattgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctctagcagcttgggcacccagacctacatctgcaacgtgaatcacaagccagg

SEQ ID NO: 3 - protein, 118 aa, Homo sapiens
  misc_feature (118)..(118): Xaa can be any naturally occurring amino acid
QVQLQESGGGLVQPGGSLRLSCEASGYTFSNYWMHWVRQPPGKGLVWVSRINEDGSITNDADSVKGRSTISRDNAKNTLYLEMNSLRAEDTAVYYCTRDIGGRDAHWGQGTLVTVSSX

SEQ ID NO: 4 - protein, 219 aa, Homo sapiens
ELVMTQSPLSLPVTPGEPASISCRSSQSLLHSNGYNYLDWYLQKPGQSPQLLIYLGFNRASGVPDRFSGSGSGTDYTLKISRVEAEDVGVYYCMQGLQTPRTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 5 - protein, 217 aa, Homo sapiens
MAEVQLVQSGGGLVQPGGSLRLSCTASGFIFNNYAMSWVRQAPGKGLEWVSGISTGGSSTYHADSVKGRFTISRDNFKKTLWLQMNSLTPEDAAVYYCARHANFWNGYLEKGAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPG

SEQ ID NO: 6 - protein, 215 aa, Homo sapiens
ELQMTQSPSTLSASVGDRVTITCRASHSVSGWLAWYQQKPGKAPKLLSYEPSSLESGVPSRFSGSGSGTEFTLTISSLQPEDFATYYCQESYRITSLTFGGGTKVETRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC